(19) United States
(12) Patent Application Publication
(10) Pub. No.: US 2020/0383846 A1
(43) Pub. Date: Dec. 10, 2020

(54) ABSORBENT ARTICLE AND A METHOD OF PRODUCING ABSORBENT ARTICLE

(71) Applicant: Essity Hygiene and Health Aktiebolag, GÖTEBORG (SE)

(72) Inventors: Malin LUNDMAN, GÖTEBORG (SE); Shabira ABBAS, GÖTEBORG (SE); Filip NYLANDER, KUNGSBACKA (SE); Gunnar WESTMAN, HÄRRYDA (SE)

(73) Assignee: Essity Hygiene and Health Aktiebolag, GÖTEBORG (SE)

(21) Appl. No.: 16/769,981

(22) PCT Filed: Dec. 7, 2018
(86) PCT No.: PCT/EP2018/083942

§ 371 Date: Jun. 4, 2020

(30) Foreign Application Priority Data
Dec. 8, 2017 (EP) PCT/EP2017/082043

(51) Int. Cl. A61F 13/53; A61F 13/84; A61F 13/47; A61F 13/49; A61F 13/15; A61F 13/00; A61L 15/26; A61L 15/42

(52) U.S. Cl. CPC A61F 13/53; A61F 13/8405; A61F 13/47; A61F 13/49; A61F 2013/530817; A61F 13/00017; A61L 15/26; A61L 15/425; A61F 13/15723

(57) ABSTRACT

An absorbent article and a method of manufacturing the absorbent article including an absorbent body and a foam component having a solid open-cell structure, wherein the solid phase in the foam includes cells walls including polyurethane. The polyurethane includes a reaction product of an isocyanate or an isocyanate equivalent and a polyol-hemicellulose mixture. The hemicellulose is present in the mixture in an amount of from 5 to 80% by weight, based on the total weight of the polyol-hemicellulose mixture. The hemicellulose is in the cell walls of the foam. Also, the foam component applied to a carrier.

### TECHNICAL FIELD

The present disclosure relates to an absorbent article comprising a polyurethane foam component having a solid open cell structure. The present disclosure also relates to a method of manufacturing the absorbent article comprising the foam component.

### BACKGROUND

For absorbent articles, such as diapers, incontinence products or sanitary napkins, there are high requirements relating to the products being soft and comfortable in use and that they can manage to handle relatively large amounts of bodily fluids, such as urine and/or menstrual fluid. The fluids have different viscosities and require therefore different properties from the materials used in the absorbent articles. At the same time there is a need that the products give the user a feeling of secureness and a visual impression that the bodily fluids are absorbed by the absorbent article. Therefore, the articles often contain several layers of materials to adapt the articles better to the aimed uses.

Foam materials have been used in absorbent articles to for example improve liquid handling properties, e.g. liquid receiving properties, and to increase the comfort of the articles. Example of an article containing a foam layer is disclosed e.g. in WO2014098679. The foam layers may comprise a thermoplastic foam or thermosetting foam and can be chosen from a wide variety of polymeric foams.

Continuous efforts have been made to decrease the environmental load of the absorbent articles while the comfort of the articles is improved. However, there is still room for improvements and there is a desire to make the absorbent articles more environmentally friendly.

### SUMMARY

Absorbent articles containing a foam component can often feel comfortable in use. The foam component can be polyurethane based, since polyurethane is soft, pliable and flexible, thereby rendering the product comfortable. While polyurethane is comfortable, it also has proper liquid handling properties for use in for example personal hygiene articles, such as diapers, sanitary napkins or incontinence pads or even in wound care products. Therefore, it would be desirable to use polyurethane foam in absorbent articles. However, polyurethane (PU) foam is produced by reacting isocyanate with a polyol in the presence of catalyst and/or UV light. Polyols are generally petroleum based, and there is a desire to reduce the use of petroleum-based materials.

The objective of the present disclosure is thus to provide an absorbent article comprising a polyurethane foam component which is more environmentally friendly than previously known articles containing polyurethane foam materials. It has been noted that the environmental load can be reduced by reducing the amount of petroleum-based raw materials in the foam material.

A further objective of the present disclosure is to provide absorbent article with a polyurethane foam component, which can be easily manufactured and has adequate liquid handling properties. Further, it is an objective to provide an absorbent article which is comfortable in use. Another objective is to provide an absorbent article with a foam component which can be used as a liquid acquisition layer, a storage layer and/or a distribution layer in absorbent articles. Thus, it is an objective to provide an absorbent article with a foam component which has the capacity to quickly receive liquid, distribute it in the structure and store it. Furthermore, it is an objective to provide an absorbent product with improved hydrophilicity and thus e.g. improved liquid uptake.

The objectives above are obtained by an absorbent article comprising a foam component and a method for manufacturing the absorbent article as defined in the appended claims.

The absorbent article of the present disclosure comprises a renewable raw material which at least partly replaces a petroleum-based raw material in PU-foam. Thus, less petroleum-based raw materials than in the prior art foam-containing absorbent articles is needed. The absorbent article comprises an absorbent body and a foam component having a solid open-cell structure. The solid phase in the foam comprises cells walls comprising polyurethane. According to the present disclosure, the polyurethane comprises a reaction product of an isocyanate or isocyanate equivalent and a polyol-hemicellulose mixture. Thus, at least a portion of the polyol is replaced with hemicellulose, whereby the amount of petroleum-based raw materials can be reduced, while the absorbent article is comfortable is use. The hemicellulose is suitably present in the mixture in an amount of from 5 to 80% by weight, based on the total weight of the polyol-hemicellulose mixture. The hemicellulose is comprised in the cell walls of the foam. The foam component is used in the absorbent article whereby the absorbent article obtains reduced amount of petroleum-based raw material, and is more environmentally friendly. The foam has similar properties as conventional polyurethane (PU) foams and can thus be used as a liquid acquisition layer, a storage layer and/or a distribution layer in absorbent articles. Furthermore, the foam component is soft and flexible and thus comfortable in use.

The hemicellulose may be present in the mixture in an amount of up to and including 70%, optionally up to and including 50% by weight, based on the total weight of the polyol-hemicellulose mixture. The higher the content of hemicellulose, the less hydrophobic the foam becomes. The hemicellulose may be present in the mixture in an amount of at least 5% by weight or from 10% by weight. Therefore, it is possible to control the liquid retaining properties of the foam component.

The hemicellulose may comprise at least one of xyloglucan, glucomannan, mannan, xylan, arabinoxylan, arabinogalactan, glucuronoxylan, which all are common hemicelluloses and can be easily obtained for example from wood products or from cereals, such as grain shells.

The hemicellulose may be distributed throughout the cell walls of the foam as evaluated by means of a confocal laser scanning microscopy (CLSM). Since the hemicellulose is distributed throughout the cell walls, the characteristics of the foam material will be equal throughout the whole material.

The pore radius of the foam may be from 1-500 μm, defined as the longest extension of the open cell in the X-Y plane as visible in an Environmental Scanning Electron Microscopy (ESEM) image. Also, the foam may exhibit a pore volume distribution, measured by PVD in n-hexadecane, in the pore radius range 5-425 μm. Such foam is useful as it has larger voids that may give better liquid transportation and smaller voids that have better retention properties. A high content of fine pores increases the capability of trapping large amounts of liquid, which in turn results in a good rate of absorption and wicking, which may be desirable in certain types of absorbent products.

The foam may have a free swell capacity (FSC) value of from 8-30 g/g, as measured by the standard test NWSP 240.0.R2 (15). The foam may have a retention capacity (CRC) as determined by a Centrifuge Retention Capacity Test of 0.5 to 15 g/g, as measured by the standard test NWSP 241.0.R2 (15). Thus, the foam of the present disclosure may have improved retention capacity compared to conventional PU-foams.

The foam may have a contact angle of below 100°, measured according to TAPPI method T558PM-95 (1995) at a time interval from 0.05 to 10.06 s. Thus the foam according to the present disclosure is less hydrophobic than a PU-material containing no hemicellulose.

To further improve the comfort of the absorbent article, the foam component may comprise a softener as an additive.

The foam component may also be applied on a carrier. The carrier may be a fibrous layer and the foam component may be integrated into the fibrous structure. The fibers may be cellulose fibers, synthetic fibers or a combination thereof. The fibrous carrier layer shown in FIG. 11 to FIG. 16 is a nonwoven layer with a surface weight of 150 g/m² having 20-25 weight % of bicomponent fibers of PP/PET and 75-80 weight % fibers of PET. One advantage having the foam component applied on a carrier is to increase the strength.

The foam component may also comprise Microfibrillated Cellulose (MFC) and/or Nanofibrillated Cellulose.

The term “nanofibrils” means individual fibrils having a diameter equal to or less than 100 nm at all points along the nanofibril. The practical lower limit 5 for the fiber diameter is approximately 1 nm. The diameter may vary along its length. The nanofibrils may exist as individual fibres and/or as clusters of nanofibrils. The term “nano fibrillated cellulose (NFC)” is used interchangeably with the term “nanofibrils”.

The term “microfibres” means individual fibres having a diameter equal or greater than 100 nm but less than or equal to 100 μm at all points along the microfibre. More specifically, the microfibres may have a diameter greater than 100 nm but less than or equal to 10 μm or a diameter greater than 100 nm but less than or equal to 1 μm. The diameter may vary along the length of the microfibre. The microfibres may exist as individual microfibres and/or as clusters of microfibres in the composite. The term MFC (microfibrillated cellulose) is used interchangeably with the term “microfibers”. Microfibrillated cellulose may comprise a fraction of nanofibrils.

According to an embodiment, the absorbent article may be a sanitary napkin, incontinence pad or a diaper further comprising a liquid permeable topsheet and a liquid impermeable backsheet, wherein the absorbent body and the foam component are enclosed between the topsheet and the backsheet. The absorbent body may comprise a liquid inlet material and the foam component may be comprised in the liquid inlet material being arranged in direct or indirect contact with the absorbent body, the liquid inlet material being located between the absorbent body and the liquid pervious topsheet. The foam component is suitable for the functionality as a liquid inlet layer or liquid distribution layer in an absorbent article due to the open cell structure, which allows the fluid to be distributed evenly into the absorbent body.

Alternatively, the absorbent article may be a wound care product for absorbing bodily fluids, such as blood and/or exudates. The foam component may function in such products also as a liquid inlet layer and/or as a layer to absorb shocks.

The objectives mentioned above are also attained by a method of producing an absorbent article comprising the steps of:
a) Providing a foam component by a method comprising the steps of:
   i. Dissolving hemicellulose in a solvent and providing a hemicellulose suspension;
   ii. Mixing the hemicellulose suspension and a polyol and providing a hemicellulose and polyol mixture, wherein the amount of hemicellulose is from 5 to 80% by weight, based on the total weight of the polyol-hemicellulose mixture;
   iii. Adding a catalyst and optionally one or more additives to the hemicellulose and polyol mixture;
   iv. Drying the mixture obtained from step ii) or iii) such that the water-content is below 20% by weight, preferably from 2-15% by weight
   v. Bringing the mixture from step iv) to a pre-determined temperature;
   vi. Adding an isocyanate or an isocyanate equivalent to the mixture from step v) and perform mixing;
   vii. Reacting the mixture from step vi) to provide a foam;
   viii. Stabilizing the foam; and
   ix. Cutting the foam to provide the foam component; and
b) Providing an absorbent body and optionally additional components for the absorbent article;
c) Assembling the absorbent body, the foam component and the optional additional components together to provide the absorbent article.

According to the present disclosure are step (i) and step (ii) carried out prior to step (iv). Otherwise, the mutual order between step (i), step (ii), step (iii) and step (iv) may vary.

The method can be performed in existing assembly equipment for absorbent articles, whereby there is no need for expensive investments. The foam component has also similar mechanical properties as conventional PU-foam components, which is an advantage in the manufacturing process.

The method may further include the step of adding a surface active agent, which is silicone oil, to the hemicellulose and polyol mixture in the step iii). In this way, the foam generating properties may be improved, while the foam obtains desirable properties.

The method may further include a step of adding a softener to the hemicellulose and polyol mixture in the step iii). The softener may affect the mechanical properties of the foam.

In the method the pre-determined temperature in the step v) may be from 10 to 50° C., and wherein the isocyanate or isocyanate equivalent added in the step vi) has the same or higher temperature. In this way, the step may be performed in ambient conditions.

The isocyanate may be a di-isocyanate and may have an index value of from 100-110, calculated as the ratio actual weight:theoretical weight, the ratio multiplied with 100. In this way proper yield is obtained.

In the step b) additional components including a liquid permeable topsheet and a liquid impermeable backsheet may be provided to the absorbent article. In the step c) the absorbent body and the foam component may be enclosed between the topsheet and the backsheet.

The method may further include a step of transfer the mixture to a carrier in the step vi). In the method including a carrier, the method does not necessarily include step ix) cutting the foam to provide the foam component. However, when making the foam, the outer surface of the foam may form a thin liquid impermeable film layer without open pores, and therefore it may be an advantage to take away this outer thin film layer, for example by cutting, to improve the liquid inlet into the foam component.

The method may further include to add Microfibrillated Cellulose to the component by in step i) dissolving hemicellulose in an aqueous dispersion of Microfibrillated Cellulose (MFC) and providing a hemicellulose suspension including the Microfibrillated Cellulose (MFC).

The present disclosure also relates to an absorbent article produced by the method described above.

Further features and advantages of the present absorbent article are described below with reference to the detailed description and appended drawings.

### BRIEF DESCRIPTION OF THE DRAWINGS

FIG. 1 shows schematically an open diaper from a side view as an example of an absorbent article according to the present disclosure.

FIG. 2 shows schematically the layers of the diaper of FIG. 1 in a transversal cross-section.

FIG. 3a illustrates by means of an environmental SEM a 150× magnification of the structure of a foam component having 0% hemicellulose replacing polyol.

FIG. 3b illustrates a 1000× of the same foam component as illustrated in FIG. 3a.

FIG. 4a illustrates by means of an environmental SEM a 150× magnification of the structure of a foam component having 10% hemicellulose replacing polyol.

FIG. 4b illustrates a 1000× of the same foam component as illustrated in FIG. 4a.

FIG. 5a illustrates by means of an environmental SEM a 150× magnification of the structure of a foam component having 20% hemicellulose replacing polyol.

FIG. 5b illustrates a 1000× of the same foam component as illustrated in FIG. 5a.

FIG. 6a illustrates by means of an environmental SEM a 150× magnification of the structure of a foam component having 30% hemicellulose replacing polyol.

FIG. 6b illustrates a 1000× of the same foam component as illustrated in FIG. 6a

FIG. 7a illustrates by means of an environmental SEM a 150× magnification of the structure of a foam component having 50% hemicellulose replacing polyol.

FIG. 7b illustrates a 1000× of the same foam component as illustrated in FIG. 7a.

FIG. 8 illustrates a CLSM with fluorescence images in which hemicellulose, which is included in different contents, is labelled.

FIG. 9 illustrates the pore volume distribution with respect to the pore radius.

FIG. 10 illustrates contact angle as a function of time of a reference material of PU-foam and a PU-foam according to the present disclosure in which 50% by weight of the polyol is replaced with hemicellulose.

FIG. 11 illustrates by means of an environmental SEM a 100× magnification of the structure of a foam component having 50% hemicellulose replacing polyol integrated with a fibrous carrier layer.

FIG. 12 illustrates by means of an environmental SEM a 150× magnification of the structure of a foam component having 50% hemicellulose replacing polyol integrated with a fibrous carrier layer and a 350× magnification of the same foam component integrated with a fibrous carrier layer.

FIG. 13 illustrates 150× magnification of two different structures of foam components having 50% hemicellulose that are integrated with fibrous carrier layers.

FIG. 14 illustrates 150× magnification of the structure of a foam component having 50% hemicellulose which is integrated with a fibrous carrier layer and a 80× magnification of the same foam component integrated with a fibrous carrier layer. The foam component is a disintegrated component within the fibrous carrier and not a continuous foam layer.

FIG. 15 illustrates 150× magnification of the structure of a foam component having 50% hemicellulose. As seen in the figure the foam has an open pore structure.

FIG. 16 illustrates 80× magnification of a structure of a foam component having 25% hemicellulose and 25% Microfibrillated Cellulose (MFC).

### DETAILED DESCRIPTION

The absorbent article according to the present disclosure comprises a foam component which comprises or consists of foam built of a continuous three-dimensional network or cellular structure of a solid phase, which surrounds a gaseous phase dispersed therein. The porous foam component contains pores and cavities that are connected to each other to form a fine interconnected network. Such foam is stable both in dry and wet conditions, and does not fall apart under pressure. In the foam the solid phase is a polymeric material, which forms the cellular structure by means of cell walls in the continuous cellular phase. Thus, the cell walls constitute the solid phase of the foam. The cells may have different shapes, sizes and topographies and may be open. In the open cell structure the cells communicate with each other and delimit the cells comprising the gaseous phase therein. Therefore, the foam can be for example used as a liquid inlet layer in an absorbent product, since the liquid can easily enter the foam. The foam may comprise a minor amount of closed cells. However, by having a majority of open cells, the functionality as a liquid inlet layer or liquid distribution layer in an absorbent article can be obtained. The open-cell polymeric foam component may alternatively or additionally function as liquid absorbent. The component can be heavily compressed, and it can have an ability to swell when in contact with liquid whereby the liquid is absorbed into the cell structure of the foam. Thus, the foam may have hydrophilic property. Hydrophilicity and/or wettability are typically defined in terms of contact angle of foam and are described more in detail below. The foam structure may comprise many fine interconnected pores which may be capable of absorbing liquid.

In connection with the present disclosure by “hydrophilic” is meant that when a surface of a substrate is wetted by aqueous fluids (e.g., aqueous body fluids), the surface is said to be wetted by a fluid (i.e., hydrophilic) when the contact angle between the fluid and the surface is less than 90 degrees, as measured at 0.1 seconds using the Dynamic Absorption Test described below. Conversely, a surface is considered to be “hydrophobic” if the contact angle is greater than 90 degrees as measured at 0.1 seconds using the Dynamic Absorption Test described below.

The solid phase and thus the cell walls of the foam comprise or consist of polyurethane. Generally, the polyurethane foam can be obtained from the reaction between an isocyanate and a polyol in the presence of a catalyst. The reaction is exothermic and renders polyurethane polymer in which organic units are joined by carbamate links. The foam component used in the absorbent article of the present disclosure comprises polyurethane comprising a reaction product of an isocyanate and a polyol-hemicellulose mixture, i.e. a portion of conventional polyol has been replaced with hemicellulose. The hemicellulose can be present in the polyol-hemicellulose mixture in an amount of from 5 to 80%, or 10 to 70%, or 10 to 50% by weight, based on the total weight of the polyol-hemicellulose mixture. Polyurethane does not normally have a high absorbent capacity. However, the use of hemicellulose may decrease the hydrophobicity of the polyurethane. The higher the hemicellulose content is the less is the hydrophobicity of the material.

The hemicellulose is comprised in the solid phase and thus in the cell walls of the foam. The hemicellulose may be distributed substantially uniformly in the cell walls, which can be seen for example in an image obtained from confocal laser scanning microscopy (CLSM). By substantially uniformly is in this context meant that hemicellulose is present in the cell walls so that it is included in the structure of the polyurethane. However, the amount of hemicellulose in different cell walls may vary, but hemicellulose is not included only as aggregates of hemicellulose in the material. Thus, hemicellulose may be chemically bound to the polyurethane. The method for manufacturing such foam is described more in detail below.

The present obtained polymeric foam is suitably pliable and flexible, meaning that it can be easily bent and deformed. In this way it adapts to the body of the user of the absorbent article. Also, the foam is suitably resilient or elastic, meaning in this context that it has an ability to return to its shape when the bending or deforming force is released. In this way the material may additionally function as cushion, i.e. so that it may dampen outside forces to a certain grade and thus further improve the comfort of the absorbent article during use. The foam is also soft, which means that it yields readily to touch or pressure.

The isocyanate may be a di- or a polyisocyanate, and thus contain more than one reactive isocyanate groups (—NCO) per molecule. The isocyanate may be obtained for example from crude oil or natural gas. An non-limiting example of a suitable isocyanate is diphenylmethane 4,4′-diisocyanate (pMDI), but of course any di- or poly isocyanate with similar functionality may be used. Alternatively, isocyanate equivalents may be used, i.e. other conjugations reacting in a similar way as isocyanate and creating atom bonds, which may be non isocyanate equivalents. Such pathways include reaction of cyclic carbonates with amines, self-polycondensation of hydroxyl-acyl azides or melt transurethane methods.

The polyols are alcohols that contain multiple hydroxyl groups. Besides being essential for the formation of polyurethane, the polyols may render the foam with flexible characteristics. The polyol may also be obtained for example from crude oil or natural gas. An example of a suitable polyol is glycerol propoxylate block ethoxylate (GPE), but of course any polyol with similar functionality may be used. However. GPE has been noted to be suitable for providing the foam component usable in absorbent articles, since it includes both hydrophilic and hydrophobic groups. By hydrophilic group is meant a group having a strong affinity for water and by hydrophobic group is meant a group having no or little affinity for water.

According to the present disclosure the polyol is at least partly replaced by hemicellulose, which is a carbohydrate biopolymer. Hemicellulose is a polysaccharide that is present in almost all plant cell walls and can be obtained for example from wood products or from cereals, such as grain shells. Hemicelluloses are more complex than cellulose and can be hydrolysed to monosaccharides and other products. Examples of common hemicelluloses are thus xyloglucan, glucomannan, mannan, xylan, arabinoxylan, arabinogalactan and glucuronoxylan, which all are common hemicelluloses and can be easily obtained for example from wood products or from cereals, such as grain shells. The hemicellulose may be arabinoxylan, which has been found to be suitable for foam formation.

Catalysts used in the reaction may be any catalyst suitable for use for the polymerization reaction to obtain polyurethane. Non-limiting examples of suitable catalysts are gelling catalysts, such as organo-metal or organo-tin catalysts, e.g. dibutyltin dilaurate (DBTL), dibutyltin diacetate, dibutyltin sulfide, stannous octate, iron acetylacetonate and copper acetylacetonate. Alkali metal salts such as sodium hydroxide, potassium acetate and calcium hexanoate can be also used. Also blowing catalysts can be used, such as non-nucleophilic amines, such as tertiary amines or delayed action forms thereof, e.g. triethyl amine, triethylenediamine, bis[2-(N,N-dimethylamino)ethyl] ether, N,N,N′,N′-tetramethylethylenediamine, N,N,N′,N′-tetraethylethylenediamine, N,N,N′,N′-tetramethyl hexamethylenediamine, N,N,N′,N′-tetramethylguanidine, N,N,N′,N′-tetramethyl-1,3-butanediamine, N,N,N′-trimethylaminoethyl ethanolamine, N,N-dimethyl cyclohexylamine, N,N-dimethylethanolamine, N,N-diethylethanolamine, N-methylmorpholine and N-ethylmorpholine or delayed-action salts of that amine, or e.g. 1,4-diazabicyclo[2.2.2]octane (DABCO).

The polyurethane foam component used in the present absorbent article may include additives. The additives may include surface active agents, plasticizers and/or softeners, crosslinking agents, foam forming additives and/or additives for cell stabilization. Example of a suitable surface active agent is silicone oil, but any other surface active agent may be used. Non-limiting examples of suitable plasticizers and softeners include glycerol triacetate and alkyl citrate which are biobased plasticizer, and e.g product Geolite® from the company Momentive®, polydimethylsiloxane provided e.g. as a product name Baysilone from e.g. Momentive®, Hexamoll® from BASF® or Ortegol® from Evonik®. The additives are suitably skin-friendly and non-toxic, since they are to be used in contact with skin and in close proximity to mucous membrane.

The foam component used in the absorbent article of the present disclosure may be produced by a method including steps as further defined below.

In the first step (i) suitable hemicellulose is dissolved in a solvent. The solvent may be water or the solvent may be an organic polar solvent, e.g. an alcohol, or dimethylsulphoxide, or the solvent may be water or an aqueous solution containing water and an additional solvent, e.g, an organic polar solvent, e.g. an alcohol, or dimethylsulphoxide. The solvent is suitably an aqueous solution and may contain water in an amount of from 0.1 to 100% by weight, the balance containing another solvent, e.g. as mentioned above. The hemicellulose is at least partly dissolved whereby a hemicellulose suspension is provided. Alternatively, if the hemicellulose is completely dissolved, a hemicellulose solution is provided. The suspension or solution may be heated to an elevated temperature, and e.g. in case the solvent comprises or consists of water it can be heated to a temperature just below the boiling point of the solvent. For example, if the solvent is water, the suspension/solution may be heated to just below 100° C., for example to a temperature of from 50-90° C. In this way the hemicellulose may be further dissolved to the solvent and the suspension may become clear. By suspension is meant in this context a homogenous dispersion which may contain small aggregates, which are not sedimented, and dissolved hemicellulose. The mixture may alternatively be a solution in which the hemicellulose is completely dissolved. Since the suspension and the solution are homogen, it is possible to provide foam in which the hemicellulose is contained more homogenously in the cell walls of the foam. The aqueous hemicellulose suspension or solution is then provided for further steps in the method.

In the next step (ii) the aqueous hemicellulose suspension or solution and a polyol are mixed, and a hemicellulose and polyol mixture is provided. The amount of hemicellulose in the mixture may vary from 5 to 80% or from 10 to 70% or from 10 to 50% by weight, based on the total dry weight of the hemicellulose polyol mixture. In the corresponding way, the amount of polyol may vary from 20 to 90% or from 30 to 90% or from 50 to 90% by weight, calculated as the total dry weight of the hemicellulose polyol mixture. Thus a hemicellulose and polyol mixture is provided. The mixture may be dried at this stage or later on if water is present in the solvent. The amount of water may be reduced to an approximate content of below 50% by weight of the mixture, and can be for example from 1 to 20% by weight or from 4 to 10% by weight. The amount of water affects the size of the pores in the foam and generally the more water is present, the larger the pores.

In the next step (iii) a catalyst is added to the hemicellulose and polyol mixture. The amount of catalyst can vary from 0.2 to 5% by weight of the total weight of the hemicellulose and polyol mixture including the catalyst. According to an embodiment, two different types of catalysts may be used. For example, the first type may be a gelling catalyst, such as dibutyltin dilaurate (DBTL), and the second type may be a blowing catalyst, such as 1,4-diazabicyclo[2.2.2]octane (DABCO). The amount of the gelling catalyst may vary from 0.3 to 4.9%, for example from 0.5 to 2.5%, suitably from 0.8 to 1.2% by weight of the hemicellulose and polyol mixture. The amount of the gelling catalyst may be larger than the amount of the blowing catalyst. The amount of the blowing catalyst may vary from 0.1 to 2% or from 0.3 to 1%, suitably from 0.4 to 0.6% by weight of the hemicellulose and polyol mixture. Furthermore, one or more additives may be added at this stage. Suitable additives have been listed above and the amount of the additives may vary from 0-10% or 1 to 5%, or from 1.5 to 4% by weight of the hemicellulose and polyol mixture. If the mixture is not dried or not sufficiently dried in the previous step, it may be alternatively or additionally dried after the addition of the catalysts and/or the additives to desired water content in a step (iv), which may be within the ranges defined above. Also, the mixture can be mixed thoroughly at this stage to ensure that as homogenous mixture as possible is obtained.

In the next step (v), the hemicellulose and polyol mixture including the catalysts and the optional additives is brought to a temperature of from 10 to 50° C., which suitably corresponds to the ambient temperature. Subsequently in the next step (vi), an isocyanate or an isocyanate equivalent, such as a di-isocyanate, to be added to the hemicellulose and polyol mixture in the step (vi) may be brought to the same temperature as the hemicellulose and polyol mixture. The isocyanate may have an index value of from 80-120, suitably from 100-110, calculated as the ratio actual weight:theoretical weight, the ratio multiplied with 100. In this way the reaction may be kept stable.

In the next step (vi), the isocyanate, such as the di-isocyanate, or an isocyanate equivalent which may be a non-isocyanate, is added to the hemicellulose and polyol mixture including the catalysts and optional additives from the previous step (v) and the obtained mixture is mixed thoroughly to obtain a polyurethane foam.

In the next step (vii) the mixture from the previous step (vi) is reacted to provide foam. The foam may be free rise foam, also referred to as slabstock foam. There are several different methods for foam generation or to further improve foam generation such as mechanical agitation, air injection, heating, gas generation, evaporation, enzymatic decomposition and phase separation techniques, and the methods per se and conditions required are known to the skilled person. Any of the known methods may be used, and these foam generation methods may be used especially in case the solvent is water-free, but can of course be used also in case the solvent is aqueous.

After the reaction, the foam may be washed if other solvent than water is used. Subsequently, the foam may be stabilized in a step (viii) or the foam may be stabilized before washing. The stabilization may be performed by letting the foam rest for a pre-determined or desired time period. For example, stabilization may include letting the foam rest for at least 24 h at a temperature of from 10 to 30° C., but is not limited thereto.

In the next step (ix) the foam is cut to a desired shape to provide the foam component suitable for the absorbent article of the present disclosure. When manufacturing an absorbent article, the method further comprises providing an absorbent body and optional additional components for the absorbent article and assembling the absorbent body, the foam component and the optional additional components together to provide the absorbent article. The assembly of the absorbent article is adapted to the absorbent article in questions and such methods are known to the skilled person and not described in detail herein.

The foam component is porous and as used herein, the term “porous” refers to a material comprising pores and which admits the passage of gas or liquid through these pores. In addition to the liquid absorption properties, the absorbent article of the present disclosure also may have a certain liquid retention capacity. The liquid retention capacity (CRC) may be determined by the Centrifuge Retention Capacity Test. The CRC of the present foam component may be higher than that of a conventional polyurethane foam component and may vary from 0.5 to 15 g/g, suitably from 0.5 to 8 g/g. Hence, the foam has the ability to trap and retain a certain amount of liquid within the pores and cavities of the foam, whereby the total absorption capacity of the absorbent article may be increased.

The absorbent porous foam may exhibit a pore volume distribution, measured by PVD in n-hexadecane, in the pore radius range 5-425 μm. Such foam is useful as it has both larger voids that may give better liquid transportation and smaller voids that have better retention properties. A high content of fine pores increases the capability of trapping, which in turn results in a good rate of absorption and wicking. The pore volume may be controlled by means of the manufacturing process, e.g. by the choice of the blowing catalyst or agent.

The absorbent article may be a sanitary napkin, incontinence pad or a diaper further comprising a liquid permeable topsheet and a liquid impermeable backsheet, wherein the absorbent body and the foam component are enclosed between the topsheet and the backsheet. Furthermore, the absorbent article may be a wound care product.

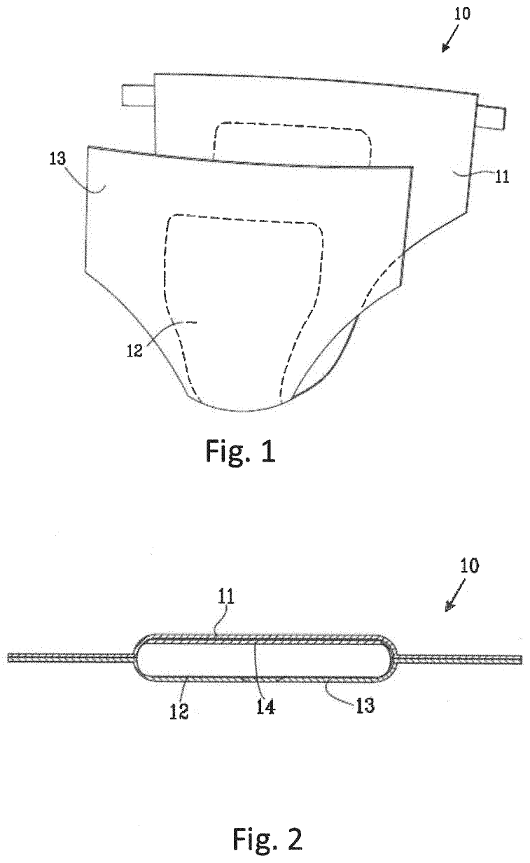

An example of an absorbent article 10 is shown in FIG. 1. The example shown is in the form of an open diaper. However, other types of absorbent articles could be sanitary napkins, panty liners, and incontinence protection articles such as incontinence pads. Also, the absorbent article could be a wound care product (not shown). The absorbent article 10 of the present disclosure typically comprises a liquid-permeable topsheet 11, a backsheet 13 and an absorbent body 12 enclosed between the liquid-permeable topsheet 11 and the backsheet 13. The foam component is present in the absorbent article, and in the illustrated example the foam component constitutes a liquid inlet layer 14 placed between the topsheet 11 and the absorbent body 12. The liquid permeable topsheet 11 faces the wearer's body during use and is arranged to absorb body liquids such as urine and blood. The material of the topsheet 11 may e.g. be a nonwoven material of spunbond type, a meltblown material etc. The backsheet 13 is typically liquid-impermeable, optionally breathable and may e.g. be a plastic (e.g. polyolefin) film, a plastic coated nonwoven or a hydrophobic nonwoven.

The absorbent body 12 acts to receive and contain liquid and other bodily exudates. As such, it may contain the foam component inside the absorbent body or the foam component may be placed between the absorbent body and the topsheet as illustrated in FIG. 2 and may thus act as a liquid acquisition layer or a liquid distribution layer. The absorbent article may contain additional absorbent materials. Examples of commonly occurring absorbent materials are cellulosic fluff pulp; tissue layers; superabsorbent polymers; other types of absorbent foam materials, absorbent nonwoven materials or the like. The absorbent body 12 may be constructed from several layers, such as the liquid acquisition or distribution layer or a storage layer in order to fulfil the functions which are desired with an absorbent body; i.e. capacity to quickly receive liquid, distribute it within the body and store it. The layers of the absorbent body 12 are designed to receive a large amount of liquid in a short time and distribute it evenly across the absorbent body. The foam component of the present disclosure may be present in one or more such layers, and even in all layers. The size and absorbent capacity of the absorbent body 12 may be varied to be suited for different uses such as for baby diapers, sanitary napkins and incontinence pads.

FIG. 2 is a transverse cross-sectional view of an absorbent article 10, such as the diaper shown in FIG. 1; through the mid-point of the article. It shows a liquid-permeable topsheet 11, a backsheet 13 and an absorbent body 12 enclosed between the liquid-permeable topsheet 11 and the backsheet 13. In the embodiment illustrated in FIG. 2; the foam component 14 is placed between the absorbent body 12 and the topsheet 11. In other embodiments; at least one layer of the absorbent body may comprise one or more foam components e.g. as fractions mixed with a primary absorbent material of the absorbent body; such as fluff pulp. The primary absorbent material may be a conventional material used in an absorbent body, e.g. cellulosic fluff pulp, tissue layers, absorbent foam materials, absorbent nonwoven materials or superabsorbent polymers (SAP). Accordingly, the foam component may be present in the form of a sheet or the foam component 14 may be cut into smaller fractions or pieces, which are applied in localized areas of the absorbent body. When such fractions are mixed with the primary absorbent material, e.g. a material comprising superabsorbent polymer(s); the spreading and wicking of the liquid within the absorbent body or layer(s) thereof may be improved. This has the advantage that liquid is more efficiently spread within the absorbent body or a layer thereof.

### EXAMPLES

### Example 1

Sample foam components were manufactured according to the method described below with formulations as shown in Table 1. The values are given as weight equivalents. The isocyanate index corresponds to ratio actual weight/theoretical weight multiplied with 100, and the isocyanate is diphenylmethane 4,4′-diisocyanate (pMDI). The abbreviations in the Table 1 correspond to:

AX is arabinoxylan;

GPE: glycerol propoxylate-block-ethoxylate (Mn 4000),

pMDI: polymeric methylene diphenylene diisocyanate;

DBTL: dibutyltin dilaurate;

DABCO: 1,4-diazobicyclo[2.2.2]octane;

tegostab: Evonik Tegostab® B 8040.

TABLE 1
Foam formulations

| | | | Iso- | | | | |
|---|---|---|---|---|---|---|---|
| | | | cyanate | | | | Silicone |
| Name | | | (pMDI) | | | | oil |
| (type) | GPE | AX | index | DBTL | DABCO | H₂O | (name) |
| PU | 100 | 0 | 105 | 1 | 0.5 | 4 | 2 |
| GPE | | | | | | | (tegostab) |
| PU | 90 | 10 | 105 | 1 | 0.5 | 4 | 2 |
| GPE- | | | | | | | (tegostab) |
| AX | | | | | | | |
| PU | 80 | 20 | 105 | 1 | 0.5 | 4 | 2 |
| GPE- | | | | | | | (tegostab) |
| AX | | | | | | | |
| PU | 66 | 33 | 105 | 1 | 0.5 | 4 | 2 |
| GPE- | | | | | | | (tegostab) |
| AX | | | | | | | |
| PU | 50 | 50 | 105 | 1 | 0.5 | 4 | 2 |
| GPE- | | | | | | | (tegostab) |
| AX | | | | | | | |

### First Part (A)

A carbohydrate biopolymer, in this case arabinoxylan from barley husk, is mixed with water in a vessel. The biopolymer in water mixture was heated to 80° C., to form a clear mixture. To the biopolymer in water mixture a polyol glycerol propoxylate block ethoxylate (GPE) is added and stirred to form a uniform liquid phase.

The new polyol-biopolymer-water mixture is evaporated to give the right water amount needed for foaming. Additives are added to the mixture, the proportions used are the same as in conventional two part polyurethane foaming formulations. Conventional catalysts (gelling catalyst and blowing catalyst) are added to the mixture. The gelling catalyst is conventionally dibutyltin dilaurate and the blowing catalyst is conventionally a non-nucleophilic amine. In this case dibutyltin dilaurate and 1,4-diazabicyclo[2.2.2]octane was used. A surface active agent was added. The surface active agent is by convention silicone oil, in this case a commercial silicone oil was used. Then, the polyol-biopolymer-water additives mixture (A) is mixed thoroughly for 1 min. Finally, the first part A is brought to a specific temperature (ambient temperature used in this case).

### Second Part (B)

A di-isocyanate (B), in this case polymeric methylenediphenyl 4,4″-diisocyanate (pMDI) is brought to the same temperature as part A.

### Mixing

Part A and part B were added together and mixed in a plastic vessel for a specific time (30 s-1 min). The foam was then left to rise freely and was left for one hour before being removed from the beaker. The foam was then left to rest for 7 days before evaluation of the foam properties.

### Example 2

### Structure of the Foam (ESEM)

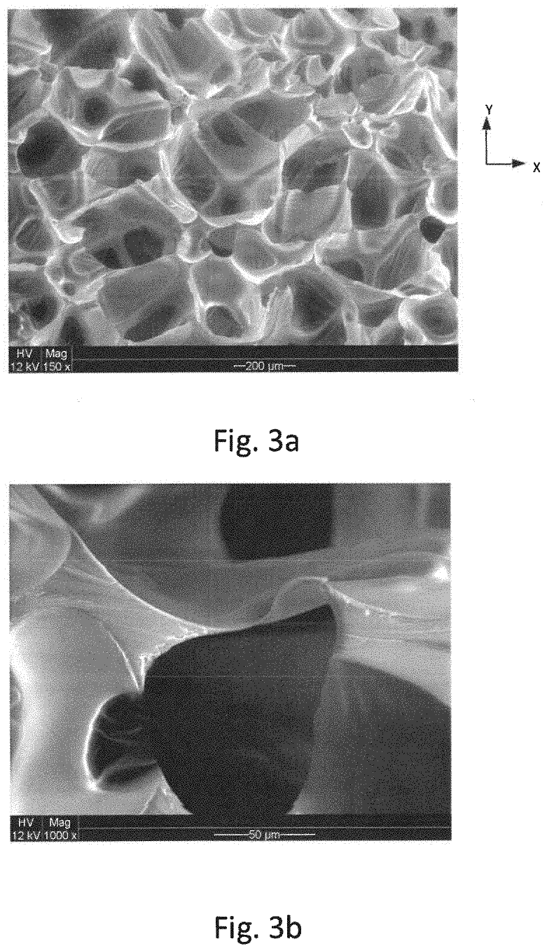

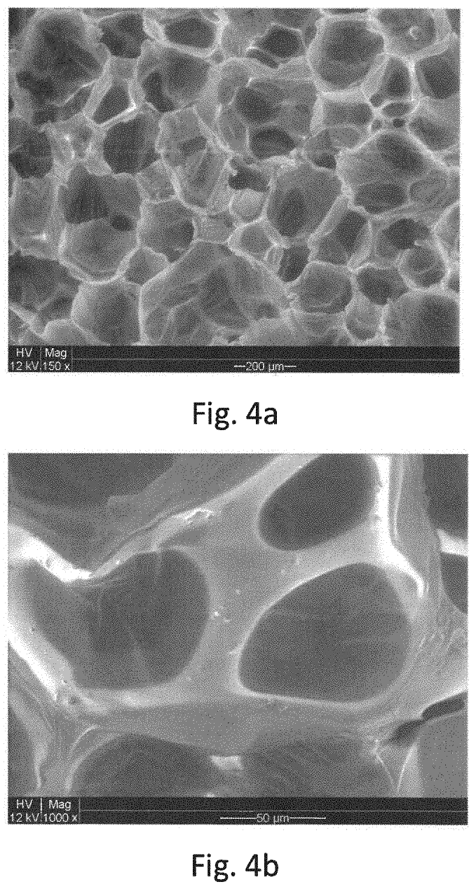

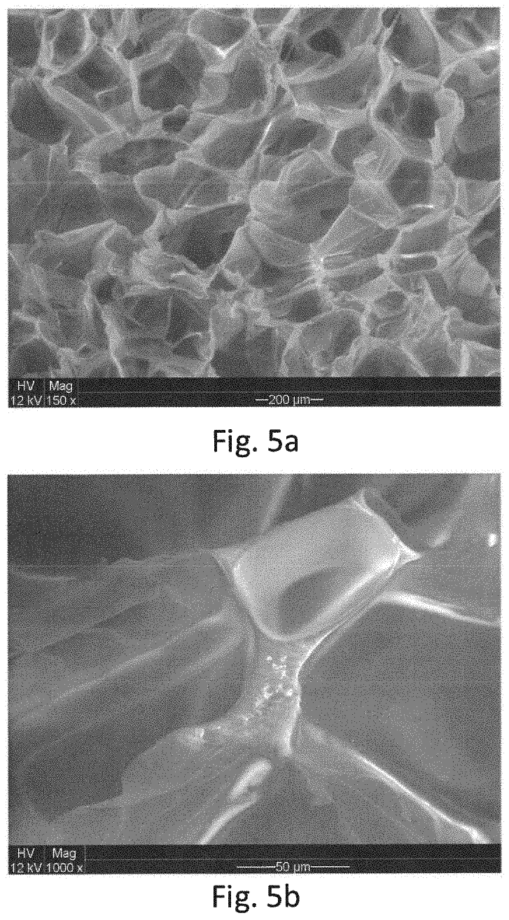

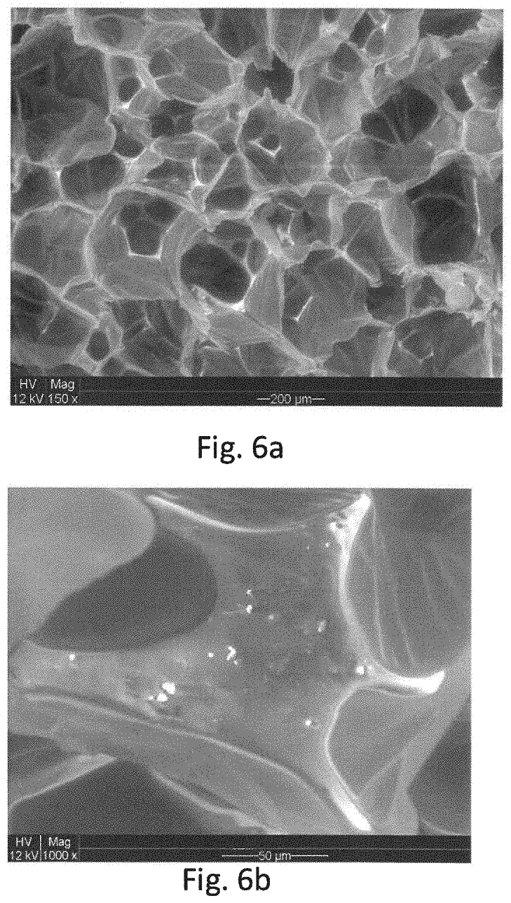

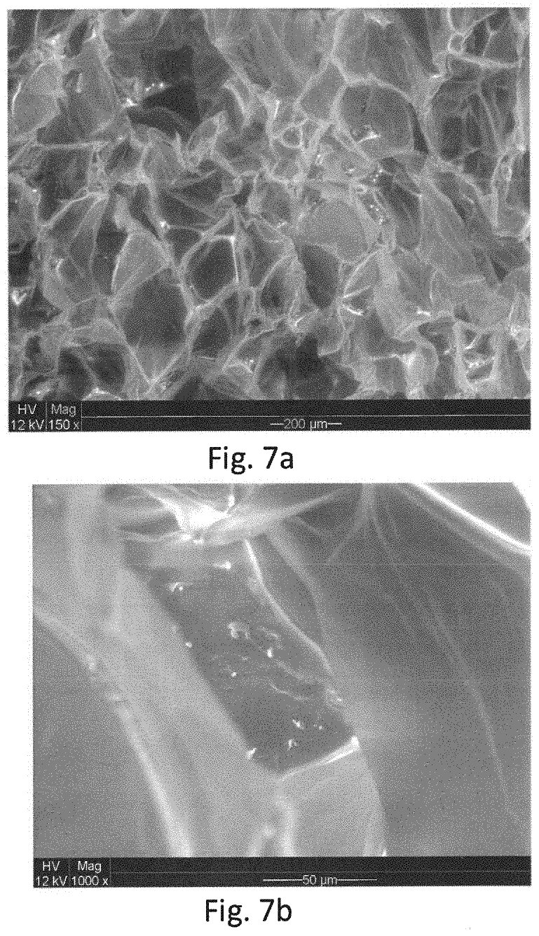

Environmental Scanning Electron Microscopy (ESEM) was used to study the structure of the samples of Table 1. FIGS. 3-3a show reference material with no arabinoxylan. The magnification is 150×, and 350×, 1000×, respectively. In FIG. 4-4a, the ratio GPE/AX is 90/10, in FIG. 5-5a, the ratio GPE/AX is 80/20, in FIG. 6-6a, the ratio GPE/AX is 66/33, and In FIG. 7-7a, the ratio GPE/AX is 50/50.

A sample was prepared by first taking out a small sample of the respective polyurethane foam. Then the surfaces of the sample were sputtered with an approximately 20 nm thick layer of gold ions with a JEOL JFC-1100E ion sputter. After the coating step, the samples stubs were placed in a JEOL JSM-820 scanning microscope at acceleration voltage of 20 kV. Digital photos of the samples were collected by the JEOL Semafore SA20 slow scan digitalizer and the Semafore 5.1 software.

From the images it can be seen that the pore radius can vary from 1-500 μm, defined as the longest extension of the open cell in an X-Y plane as visible in the ESEM image. The X-Y plane is shown in FIG. 3a and applies to all images shown in FIG. 3a-7b. Thus, the image is evaluated only in X-Y plane corresponding to normal coordinate axes and Z-dimension is not evaluated.

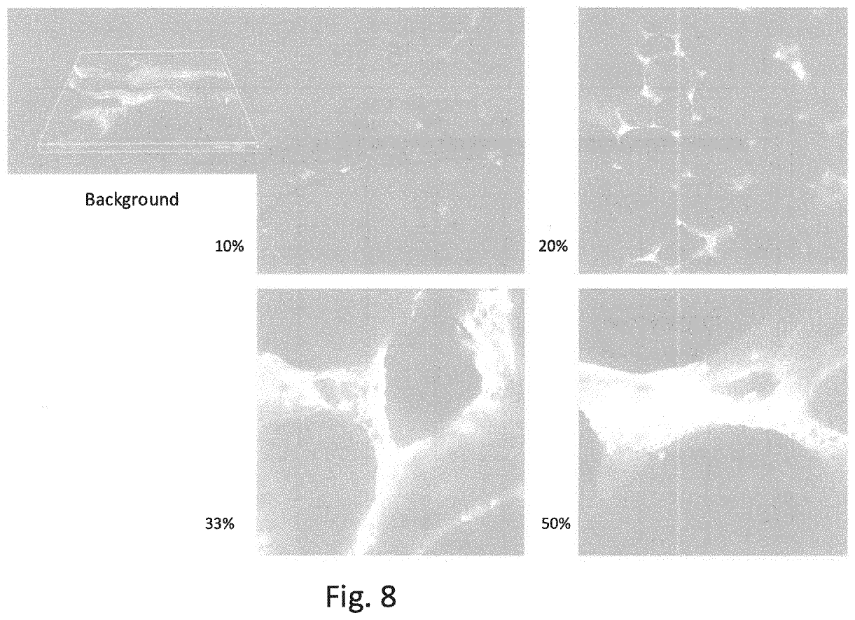

From FIG. 8 it can be seen that hemicellulose is included in the solid phase of the foam, i.e. in the cell walls of the obtained foam. The images have been obtained by means confocal laser scanning microscopy (CLSM). A Nikon Ti-E/A1+ confocal laser scanning microscope (Nikon Corporation, Minato, Tokyo, Japan) with the NIS-elements software was used, with an excitation line of 488 nm detected by a GaAsP detector. The hemicellulose (arabinoxylan) has been labelled with fluorescein isothiocyanate. In the background image no hemicellulose is included. In the other images, the hemicellulose content was 10%, 20%, 33% and 50% AX, respectively. In FIG. 8, the light portions correspond to the labelled AX, and it can be seen that the higher the AX-content, the higher is the amount of AX in the cell walls.

### Example 3

In this example pore volume distribution was determined.

### The Pore Volume Distribution (PVD) Determination Method

PVD values for samples according to the invention and for reference samples were measured using a TRI/Autoporosimeter from TRI/Princeton, 601 Prospect Avenue, Princeton, N.J., USA. The function of the equipment is described in detail in Journal of Colloid and Interface Science, 162, 163-170 (1994). The method is based on measurement of the amounts of test liquid which can be pressed out by air from a wetted porous test sample at certain pressure levels, and the result of the measurement is presented in the form of a curve in a chart where the curve illustrates the overall pore volume for each given pore radius interval.

Each pressure level corresponds to an effective (=seen as circular) pore radius according to calculation with the LaPlace equation:

R=2γ cos θ/ΔP,

where

R=effective pore radius [m]

γ=surface tension at the liquid [J/m²]

θ=receding contact angle [°]

ΔP=pressure exerted [N/m²]

In the measurements, a circular sample with an area of 25.5 cm² was placed on the membrane (Millipore 0.22 μm cat. No GSWP 09000) in the pressure chamber of the porosimeter and wetted completely. For measuring liquid, n-hexadecane (>99%, Sigma H-0255) was used. A series of rising air pressure levels was used to get the points of the curve. For each air pressure level, liquid was forced out of the pores with pore radii corresponding to the interval from the last to the present air pressure level. The liquid forced out was weighed by scales linked to the chamber via a communicating vessel, and after equilibrium was reached a new point on the PVD curve was calculated by the integrated computer.

### Wetting Angle (Used for PVD Measurements)

In the LaPlace calculation the wetting angle is needed. This is a measure of how difficult it is for the liquid to wet a test material. A drop of liquid is applied to the test material, and depending on the nature of the test material, the drop may remain lying on top of the material or be absorbed. By measuring the base (d=diameter of drop contact area) and the height (h=height of drop), the contact angle (θ=tangent between plane and drop at contact point) formed between the liquid and the material can be calculated with the aid of the following equation:

tan (θ/2)=2 h/d

For the foam materials produced according to the disclosure, and the n-hexadecane used as a measuring liquid, there is complete wetting (the liquid is absorbed) and the contact angle θ is 0, resulting in cos(θ)=1 in the LaPlace equation.

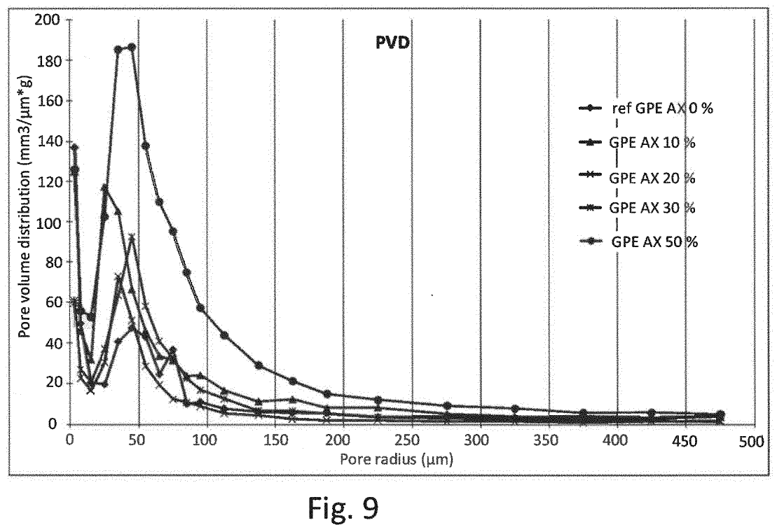

Results from the PVD measurements are shown in FIG. 9 which shows stepwise pore volume distribution for each air pressure level, corresponding to a certain pore radius, according to LaPlace equations. It can be seen that most of the pore volume is available in the pore radius range from about 10 to 150 μm for all foam materials. The reference material containing 100% polyol is indicated as ref GPE AX 0% and the examples according to the present disclosure are indicated as GPE AX 10%, GPE AX 20%, GPE AX 33% and GPE AX 50%, and correspond to the foams presented in Table 1. Thus, it can be seen that the use of hemicellulose does not affect the pore volume distribution substantially, whereby it can be used to replace conventional PU foam materials.

### Example 4

Determination of free swell capacity (FSC) and centrifuge retention capacity (CRC) is described below.

### The Test Liquid

The test liquid was 0.9% NaCl solution.

### Samples

The foam samples were cut into small pieces.

The weight of each sample was from 0.10-0.15 g.

### Free Swell Capacity (FSC)

The free swell capacity was measured by the standard test NWSP 240.0.R2 (15), wherein the step of dripping for 10 minutes has been changed to 2 minutes. The free swell capacity was also measured for 1, and 5 minutes respectively.

### Centrifuge Retention Capacity (CRC)

The Centrifuge Retention Capacity (CRC) is a measure of the fluid retention capacity (absorbent capacity) of a sample submerged in 0.9 percent NaCl saline solution for 30 minutes and then subjected to centrifugation. The centrifuge retention capacity was measured by the standard test NWSP 241.0. R2 (15). The same samples as above having the weight of 0.10-0.15 g were used for these measurements.

TABLE 2
FSC (g/g) and CRC (g/g)
Free swell capacity and Centrifuge retention capacity
NWSP 240.0.R2 (15) and NWSP 241.0.R2 (15)

| | FSC 1 min (g/g) | CRC (g/g) |
|---|---|---|
| GPE AX 0% | 16.1 | 0.4 |
| GPE AX 50% | 16.7 | 2.8 |

It can be seen that the higher the AX-content, the higher is the CRC-value. This means that the foam containing hemicellulose is less hydrophobic than the foam without hemicellulose. Thus, the foam may better retain liquid and is thus particularly suitable for absorbent products.

### Example 5

### DAT Contact Angle

The Dynamic Absorption Test (DAT) measures the absorption of a test fluid on to a sample's surface by measuring the change in contact angle of the test fluid as it makes contact with, and absorbs into the surface. A sample containing no hemicellulose (GPE AX 0%) and a sample containing hemicellulose (GPE-AX 50%) were tested,

The contact angle was determined in line with TAPPI method T558PM-95 (1995) and the apparatus used was DAT 1100 (Fibro System). The samples tested were acclimatized at 23° C. and 50% relative humidity over at least 4 hours prior to measurements. The measurements were performed in a climate-controlled room 23° C. and 50% relative humidity. The samples were present as a single layer of material and applied to a standard sample holder using double sided adhesive tapes. Parameters for the measurements were: a) liquid used was de-ionized water b) a drop volume was 5 μl c) number of drops measured for averaging the results: 25 d) in the hypothetical case where neither T558PM-95 nor the present comments address specific measurement conditions, default values as recommended by the manufacturer of the testing equipment were used. Names of suppliers of suitable testing equipment may be found in the bound set of TAPPI test methods or may be available from the TAPPI information resources centre. Preferred devices are manufactured by Fibro System AB, Stockholm and are marketed under the FibroDat® Trademark, such as FibroDat 1100 contact angle tester. iv. For those materials (e.g. hydrophilic, absorbent materials) where the contact angle varies with time, the measurement is conducted 0.05 sec after deposition of the drop. v. If it is noted that the materials to be tested lead to very high contact angles, it may become necessary to adjust the force used for releasing the drop from the syringe to prevent the drop from rolling off.

### Results

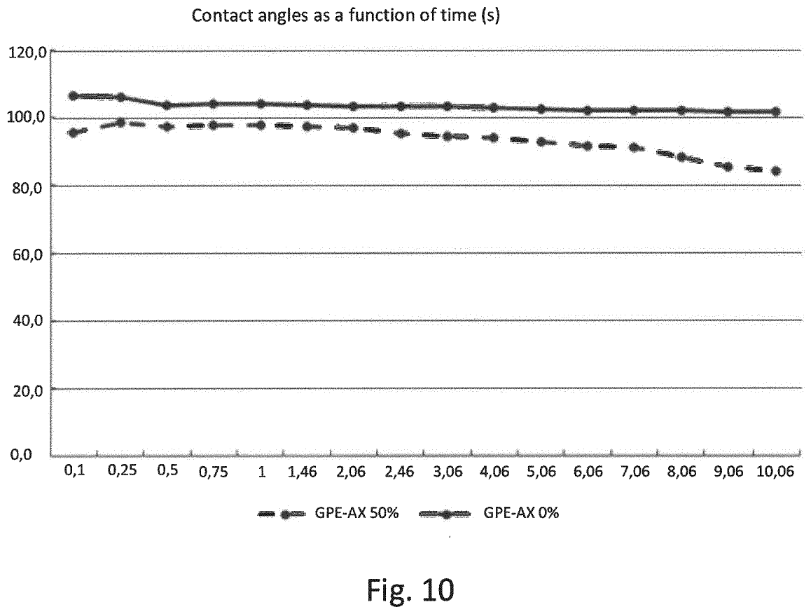

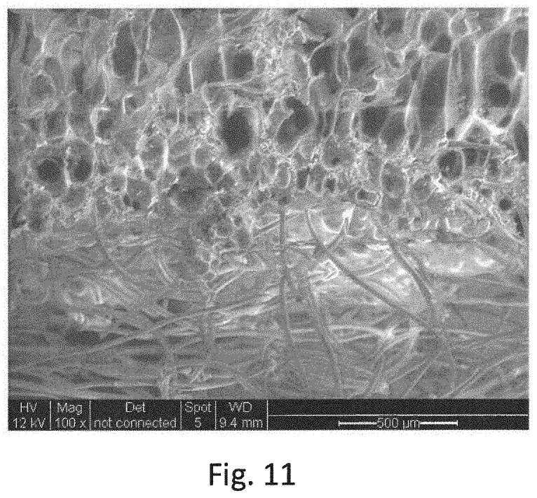

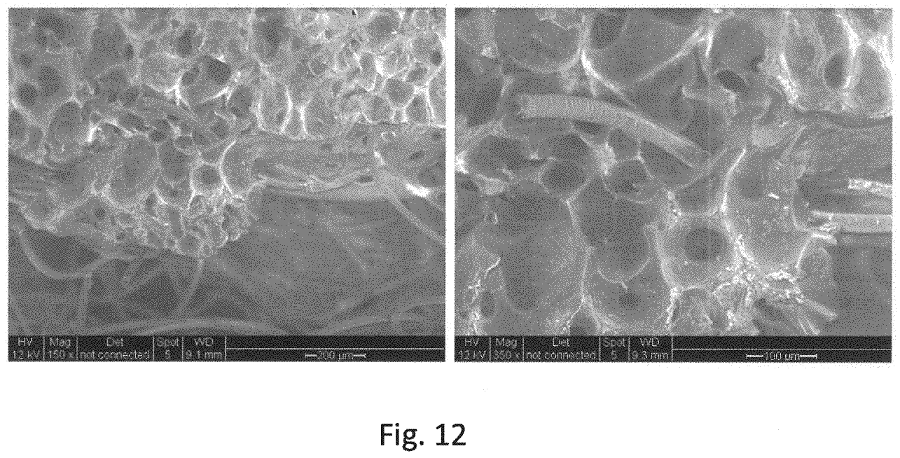

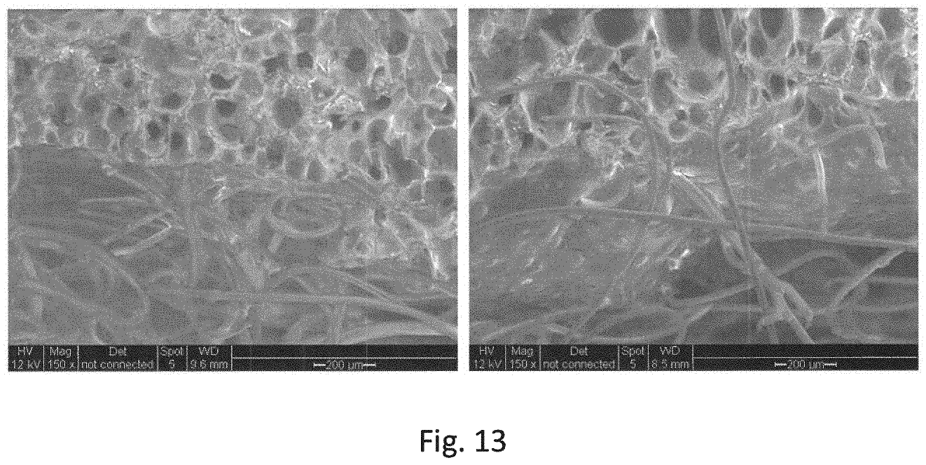

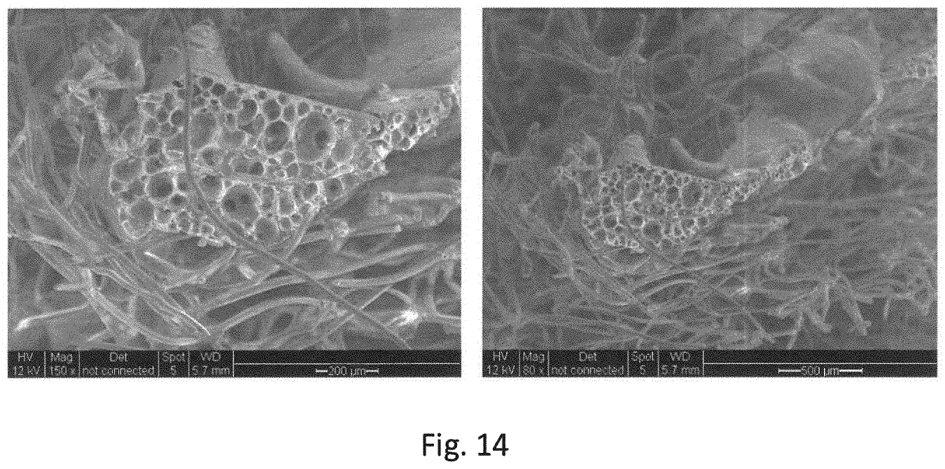

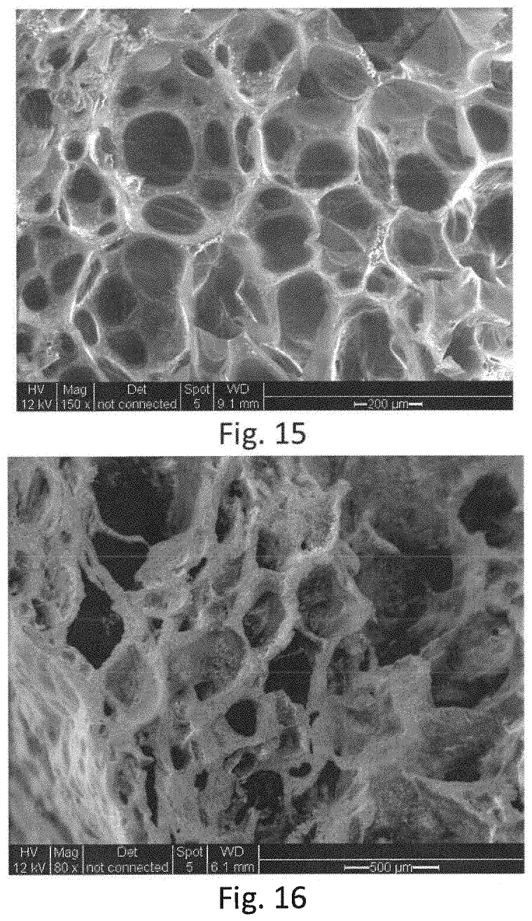

FIG. 10 illustrated an example of one drop contact angles as a function of time (y-axis in contact angle degrees and x-axis time in seconds) within a time period of from 0.05 to 10.06 s. It can be seen that the foam containing hemicellulose has overall a smaller contact angle and is below 100°, than the foam containing a conventional polyol having a contact angle of more than 100°. Therefore, the hemicellulose containing PU-foam is less hydrophobic than the PU-material containing no hemicellulose. Therefore, the foam has improved improved liquid uptake and is suitable for use in absorbent articles.

1. An absorbent article comprising an absorbent body and a foam component having a solid open-cell structure, wherein the solid phase in the foam comprises cells walls comprising polyurethane,
wherein the polyurethane comprises a reaction product of an isocyanate or isocyanate equivalent and a polyol-hemicellulose mixture,
wherein the hemicellulose is present in the mixture in an amount of from 5 to 80% by weight, based on the total weight of the polyol-hemicellulose mixture, and
wherein the hemicellulose is comprised in the cell walls of the foam.

2. The absorbent article of claim 1, wherein the hemicellulose is present in the mixture in an amount of up to and including 70% by weight, based on the total weight of the polyol-hemicellulose mixture.

3. The absorbent article of claim 1, wherein the hemicellulose comprises at least one of xyloglucan, glucomannan, mannan, xylan, arabinoxylan, glucuronoxylan, and arabinogalactan.

4. The absorbent article of claim 1, wherein the hemicellulose is distributed throughout the cell walls of the foam as evaluated by means of a confocal laser scanning microscopy.

5. The absorbent article of claim 1, wherein the pore radius of the foam is from 1-500 μm, defined as the longest extension of the open cell in the X-Y plane as visible in an Environmental Scanning Electron Microscopy image.

6. The absorbent article of claim 1, wherein the foam has a free swell capacity value of from 8-30 g/g, as measured by the standard test NWSP 240.0.R2.

7. An absorbent article according to claim 1, wherein the foam has a retention capacity as determined by a Centrifuge Retention Capacity Test of 0.5 to 15 g/g, as measured by the standard test NWSP 241.0.R2.

8. An absorbent article according to claim 1, wherein the foam has a contact angle of below 100°, measured according to TAPPI method T558PM-95 (1995) at a time interval from 0.05 to 10.06 s.

9. The absorbent article of claim 1, wherein the foam component comprises a softener as an additive.

10. The absorbent article of claim 1, wherein the polyurethane foam comprising the hemicellulose is applied on a carrier.

11. The absorbent article according to claim 10, wherein the carrier layer is a fibrous layer composed of cellulose fibers, synthetic fibers or a combination thereof.

12. (canceled)

13. The absorbent article of claim 1, wherein the absorbent article is a sanitary napkin, incontinence pad or a diaper further comprising a liquid permeable topsheet and a liquid impermeable backsheet, wherein the absorbent body and the foam component are enclosed between the topsheet and the backsheet.

14. The absorbent article according to claim 13, wherein the absorbent body comprises a liquid inlet material and wherein the foam component is comprised in the liquid inlet material being arranged in direct or indirect contact with the absorbent body, the liquid inlet foam material being located between the absorbent body and the liquid pervious topsheet.

15. The absorbent article of claim 1, wherein the absorbent article is a wound care product for absorbing bodily fluids.

16. A method of producing an absorbent article comprising the steps of:
a) providing a foam component by a method comprising the steps of:
i. dissolving hemicellulose in a solvent and providing a hemicellulose suspension;
ii. mixing the hemicellulose suspension and a polyol and providing a hemicellulose and polyol mixture, wherein the amount of hemicellulose is from 5 to 80% by weight, based on the total weight of the polyol-hemicellulose mixture;
iii. adding a catalyst and optionally one or more additives to the hemicellulose and polyol mixture;
iv. drying the mixture obtained from step ii) or iii) such that the water-content is below 20% by weight, preferably from 2-15% by weight and most preferably from 4-10% by weight;
v. bringing the mixture from step iv) to a pre-determined temperature;
vi. adding an isocyanate or an isocyanate equivalent to the mixture from step v) and perform mixing;
vii. reacting the mixture from step vi) to provide a foam;
viii. stabilizing the foam; and
ix. cutting the foam to provide the foam component; and
b) providing an absorbent body and optionally additional components for the absorbent article;
c) assembling the absorbent body, the foam component and the optional additional components together to provide the absorbent article.

17. The method according to claim 16, further including a step of adding a surface active agent which is silicone oil to the hemicellulose and polyol mixture in the step iii).

18. The method according to claim 16, further including a step of adding a softener to the hemicellulose and polyol mixture in the step iii).

19. The method according to claim 16, wherein the pre-determined temperature in the step v) is from 10 to 50° C., and wherein the isocyanate or isocyanate equivalent added in the step vi) has the same or higher temperature.

20. The method according to claim 16, wherein the isocyanate is a di-isocyanate and has an index value of from 100-110, calculated as the ratio actual weight:theoretical weight, the ratio multiplied with 100.

21. The method of producing an absorbent article comprising the steps of:
a) providing a foam component by a method comprising the steps of:
i. dissolving hemicellulose in a solvent and providing a hemicellulose suspension;
ii. mixing the hemicellulose suspension and a polyol and providing a hemicellulose and polyol mixture, wherein the amount of hemicellulose is from 5 to 80% by weight, based on the total weight of the polyol-hemicellulose mixture;
iii. adding a catalyst and optionally one or more additives to the hemicellulose and polyol mixture;
iv. drying the mixture obtained from step ii) or iii) such that the water-content is below 20% by weight, preferably from 2-15% by weight and most preferably from 4-10% by weight;
v. bringing the mixture from step iv) to a pre-determined temperature;
vi. adding an isocyanate or an isocyanate equivalent to the mixture from step v) and perform mixing; transfer the mixture to a carrier
vii. reacting the mixture from step vi) to provide a foam integrated with a carrier;
viii. stabilizing the foam integrated to the carrier; and
b) an absorbent body and optionally additional components for the absorbent article;
c) assembling the absorbent body, the foam component and the optional additional components together to provide the absorbent article.

22. (canceled)

23. The method according to claim 16, wherein in the step b) additional components including a liquid permeable topsheet and a liquid impermeable backsheet are provided, and wherein in the step c) the absorbent body and the foam component are enclosed between the topsheet and the backsheet.

24. An absorbent article produced by the method according to claim 16.

25. (canceled)
